(19) United States
(12) Patent Application Publication
(10) Pub. No.: US 2023/0129903 A1
(43) Pub. Date: Apr. 27, 2023

(54) SYSTEM AND METHOD FOR REPAIRING A LIGAMENT

(71) Applicant: Pedro Juan LOREDO, Southlake, TX (US)

(72) Inventor: Pedro Juan LOREDO, Southlake, TX (US)

(21) Appl. No.: 17/676,451

(22) Filed: Feb. 21, 2022

Related U.S. Application Data
(63) Continuation-in-part of application No. 17/509,410, filed on Oct. 25, 2021.
(60) Provisional application No. 63/286,212, filed on Dec. 6, 2021.

(51) Int. Cl. A61B 17/04; A61B 17/16

(52) U.S. Cl. CPC A61B 17/0401; A61B 17/1686; A61B 2017/045; A61B 2017/0453

(57) ABSTRACT

A surgical kit for repairing a damaged ligament includes a first anchor configured to be secured to a first bone during a surgical procedure to repair the damaged ligament. Suture tape is pre-attached to the first anchor prior to the surgical procedure and extends from the first anchor to a free end. The suture tape is configured to extend over the damaged ligament.

### CROSS-REFERENCE TO RELATED APPLICATIONS

This application is a continuation-in-part of U.S. patent application Ser. No. 17/509,410, filed Oct. 25, 2021, and claims priority under 35 U.S.C. § 119(e) to U.S. Provisional Application No. 63/286,212, filed Dec. 6, 2021, both of which are expressly incorporated by reference herein.

### TECHNICAL FIELD

The present invention is directed to a system and method for repairing a ligament and, more particularly, to a system and method of repairing a ligament using anchors.

### BACKGROUND

The hand includes multiple ligaments that attach to the joints of the finger. Injury to these ligaments is commonly due to any hard force on the finger that causes the finger to be bent too far. This force can result in a partial tear or a complete tear of the ligament. A tear to the ligament results in instability of the joint, swelling, pain, bruising, and possible deformity of the finger. Repairing the ligament involves reattaching the torn ligament with internal sutures.

### SUMMARY

The present disclosure includes one or more of the features recited in the appended claims and/or the following features which, alone or in any combination, may comprise patentable subject matter.

According to a first aspect of the disclosed embodiments, a surgical kit for repairing a damaged ligament includes a first anchor configured to be secured to a first bone during a surgical procedure to repair the damaged ligament. The first anchor includes a first inner bone anchor that is secured in an opening drilled in the first bone. Suture tape is pre-attached to the first anchor prior to the surgical procedure and extends from the first anchor to a free end. The suture tape is configured to extend over the damaged ligament. A second anchor is configured to secure the free end of the suture tape to a second bone adjacent the first bone during the surgical procedure.

In some embodiments of the first aspect, the first inner bone anchor may extend from a top opening in the first anchor to a bottom opening in the first anchor. A press may be configured to engage the first inner bone anchor through the top opening in the first anchor. The press may be configured to push the first inner bone anchor at least partially through the bottom opening in the first anchor. The first inner bone anchor may bend outward into the first bone as the first inner bone anchor is pushed through the bottom opening in the first anchor. The first inner bone anchor may include two first inner bone anchors. The two first inner bone anchors may bend outward in opposite directions into the first bone as the two first inner bone anchors are pushed through the bottom opening in the first anchor. The first inner bone anchor may include a threaded opening and the press may include a threaded screw. The threaded screw of the press is secured within the threaded opening of the first inner bone anchor. The second anchor may include a second inner bone anchor that is secured in an opening drilled in the second bone.

Optionally, in the first aspect, at least one suture may extend from the first anchor. The at least one suture may be configured to suture a first end of the damaged ligament to a second end of the damaged ligament during the surgical procedure. The second anchor may include an opening configured to receive the free end of the suture tape.

According to a second aspect of the disclosed embodiments, a method for repairing a damaged ligament includes drilling an opening into a first bone during a surgical procedure to repair the damaged ligament. The method also includes securing a first anchor to a first bone by securing a first inner bone anchor of the first anchor in the opening drilled in the first bone. The method also includes extending suture tape over the damaged ligament. The suture tape is pre-attached to the first anchor prior to the surgical procedure and extends from the first anchor to a free end. The method also includes securing the free end of the suture tape to a second bone adjacent the first bone during the surgical procedure with a second anchor.

In some embodiments of the second aspect, the method may also include engaging the first inner bone anchor through a top opening in the first anchor with a press. The method may also include pushing the first inner bone anchor at least partially through a bottom opening in the first anchor with the press. The method may also include pushing the first inner bone anchor through the bottom opening in the first anchor so that the first inner bone anchor bends outward into the first bone. The first inner bone anchor may include two first inner bone anchors. The method may also include pushing the two first inner bone anchors through the bottom opening in the first anchor so that the two first inner bone anchors bend outward in opposite directions into the first bone. The method may also include securing a threaded screw of the press within a threaded opening of the first inner bone anchor. The method may also include securing the second anchor to the second bone by securing a second inner bone anchor of second anchor in an opening drilled in the second bone.

Optionally, in the second aspect, the method may also include suturing a first end of the damaged ligament to a second end of the damaged ligament with at least one suture extending from the first anchor during the surgical procedure. The method may also include positioning the free end of the suture tape in an opening of the second anchor.

According to a third aspect of the disclosed embodiments, a surgical anchor for repairing a damaged ligament includes an anchor body having a top and a bottom. The anchor body is positioned on a bone during a surgical procedure to repair the damaged ligament with the bottom of the anchor body positioned against the bone. A top opening is formed in the top of the anchor body. A bottom opening is formed in a bottom of the anchor body. A channel extends from the top opening to the bottom opening. An inner bone anchor extends through the channel. The inner bone anchor is configured to be pushed at least partially through the bottom opening into the bone by a press that engages the inner bone anchor through the top opening.

In some embodiments of the third aspect, the inner bone anchor may bend outward into the bone as the inner bone anchor is pushed through the bottom opening in the anchor body. Suture tape may be pre-attached to the anchor body prior to the surgical procedure and may extend from the anchor body to a free end. The suture tape may be configured to extend over the damaged ligament.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a view of a surgical kit for repairing a damaged ligament including a side view of a primary anchor and a side view of a secondary anchor;

FIG. 2 is a front perspective view of the primary anchor used in the surgical kit to repair the damaged ligament;

FIG. 3 is a front elevation view of the secondary anchor used in the surgical kit to repair the damaged ligament;

FIG. 4 is a top view of a pair of pliers used to in the surgical kit to repair the damaged ligament;

FIG. 5 is a top view of a damaged ligament extending over a proximal bone and a distal bone;

FIG. 6 is a top view of the sutures of the primary anchor used to suture the damaged ligament shown in FIG. 5;

FIG. 7 is a top view of the suture tape of the primary anchor extended across the damaged ligament and inserted into the secondary anchor;

FIG. 8 is a front view of another embodiment of a surgical kit for repairing a damaged ligament including a front cross-sectional view of a primary anchor and a front cross-sectional view of a secondary anchor;

FIG. 9 is a top view of the primary anchor and the secondary anchor shown in FIG. 8;

FIG. 10 is a front view of a press configured to secure the primary anchor and the secondary anchor shown in FIG. 8 to a bone, wherein the press is in a disengaged position;

FIG. 11 is a front view of the press in an engaged position;

FIG. 12 is a front cross-sectional view of the press shown in FIG. 10 engaging one of the primary anchor or the secondary anchor shown in FIG. 8 to move an inner bone anchor to an engaged position, wherein the anchor is shown in cross-section;

FIG. 13 is a front cross-sectional view of the inner bone anchor in the engaged position;

FIG. 14 is a top view of a first bone and a second bone having a damaged ligament extending therebetween, wherein an opening is drilled into each of the first bone and the second bone;

FIG. 15 is a top view of the primary anchor shown in FIG. 8 being inserted into the first bone;

FIG. 16 is a top view of the secondary anchor shown in FIG. 8 being inserted into the second bone; and

FIG. 17 is a top view of the damaged ligament sutured with the suture of the primary anchor and the surgical tape of the primary anchor secured to the secondary anchor.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

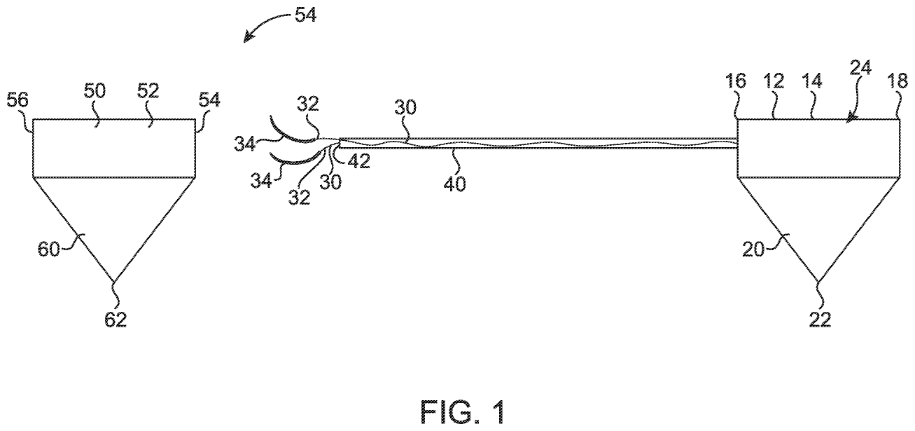

Referring now to FIG. 1, a surgical kit 10 for repairing a damaged ligament, for example a ligament in a patient's hand, includes a primary anchor 12. The primary anchor 12 incudes a generally rectangular base 14 that extends between a front end 16 and a back end 18. It will be appreciated that, in some embodiments, the primary anchor 12 may take any suitable shape, for example circular. A primary anchor tine 20 extends downward from base 14. In the illustrative embodiment, the primary anchor 12 includes a single primary anchor tine 20 that extends downward from the base 14 in a cone configuration to a point 22. In other embodiments, the primary anchor 12 may include any number of primary anchor tines 20 that extend to points 22. The primary anchor 12 also includes a top surface 24 that is configured to receive pressure from a pair of pliers 100, illustrated in FIG. 4, during a surgical procedure to repair the damaged ligament. The primary anchor tine 20 is configured to be pressed into a patient's bone by the pliers 100 during the surgical procedure so that the primary anchor 12 is secured to the patient's bone.

At least one suture 30 extends from the primary anchor 12. The at least one suture 30 extends from the base 14 to a free end 32. A needle 34 is secured to the free end 32. In the illustrative embodiment, the primary anchor 12 includes a pair of sutures 30 extending therefrom. It will be appreciated that the primary anchor 12 may include any number of sutures 30. In some embodiments, the at least one suture 30 is utilized to suture two ends of damaged ligament together during the surgical procedure to repair the damaged ligament. It will be appreciated that in some embodiments, the primary anchor 12 does not include the at least one suture 30, and the surgical procedure is performed without suturing the two ends of the damaged ligament together.

Suture tape 40 also extends from the primary anchor 12. The suture tape 40 extends from the base 14 to a free end 42. The suture tape 40 is configured to be positioned over the damaged ligament during the surgical procedure to repair the damaged ligament. The suture tape 40 is configured to increase a suture footprint allowing for increased soft tissue-to-bone contact in soft tissue repairs, e.g. ligament repairs. The suture tape 40 holds the ligament in place post-surgery to allow the ligament to reconnect to the bone. In some embodiments, the suture tape 40 is formed from a biocompatible material that allows the suture tape 40 to be permanently retained in the surgical site. In other embodiments, the suture tape 40 is formed from a material that absorbs into the body after surgery. For example, the suture tape 40 may be biodegradable and/or bio-absorbable. In some embodiments, the suture tape 40 is formed from a biocompatible material that allows tissue ingrowth. The suture tape 40 can be formed in different sizes and shapes to accommodate patient anatomy and pathology.

A secondary anchor 50 incudes a generally rectangular base 52 that extends between a front end 54 and a back end 56. It will be appreciated that, in some embodiments, the secondary anchor 50 may take any suitable shape, for example circular. A secondary anchor tine 60 extends downward from base 52. In the illustrative embodiment, the secondary anchor 50 includes a single secondary anchor tine 60 that extends downward from the base 52 in a cone configuration to a point 62. In other embodiments, the secondary anchor 50 may include any number of secondary anchor tines 60 that extend to points 62. The secondary anchor 50 also includes a top surface 64 that is configured to receive pressure from the pair of pliers 100 during the surgical procedure to repair the damaged ligament. The secondary anchor tine 60 is configured to be pressed into a patient's bone by the pliers 100 during the surgical procedure so that the secondary anchor 50 is secured to the patient's bone.

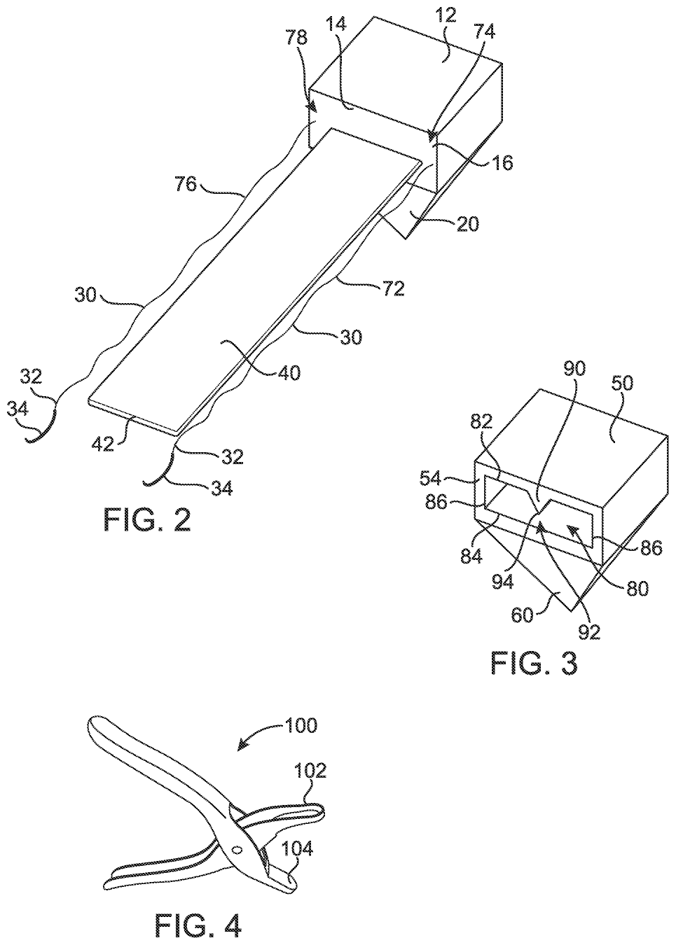

Referring to FIG. 2, in the exemplary embodiment, a pair of sutures 30 extend from the front end 16 of the base 14 of the primary anchor 12. Each of the pair of sutures 30 extends to a needle 34. The suture tape 40 also extends from the front end 16 of the base 14 of the primary anchor 12. The suture tape 40 is positioned between the pair of sutures 30. That is each of the sutures 30 is positioned on one of the sides of the suture tape 40. For example, a left suture 72 is positioned on a left side 74 of the suture tape 40, and a right suture 76 is positioned on a right side 78 of the suture tape 40. In other embodiments, the suture tape 40 and the sutures 30 may be oriented in different positions. For example, at least one suture 30 may be positioned below the suture tape 40.

Referring now to FIG. 3, the front end 54 of the secondary anchor 50 includes an opening 80 that is configured to receive the free end 42 of the suture tape 40 during the surgical procedure to repair the damaged ligament. The opening 80 is defined by a top wall 82, a bottom wall 84, and a pair of side walls 86. The secondary anchor 50 is positioned during the surgical procedure so that the front end 54 of the secondary anchor 50 faces the front end 16 of the primary anchor 12. Accordingly, the free end 42 of the suture tape 40 extends toward the secondary anchor 50. The free end 42 of the suture tape 40 is configured to be inserted into the opening 80.

An inner tine 90 extends downward from the top wall 82 toward the bottom wall 84. The inner tine 90 extends partially downward from the top wall 82 toward the bottom wall so that a slot 92 is formed between the inner tine 90 and the bottom wall 84. The inner tine 90 extends to a point 94. The slot 92 is formed between the point 94 and the bottom wall 84. The free end 42 of the suture tape 40 is configured to be inserted into the opening 80 so that the free end 42 of the suture tape 40 slides into the slot 92 under the point 94 of the inner tine 90. When pressure is applied to the secondary suture 50 to secure the secondary suture 50 to the bone, the inner tine 90 is pressed into the free end 42 of the suture tape 40 to secure the suture tape 40 in the secondary suture 50.

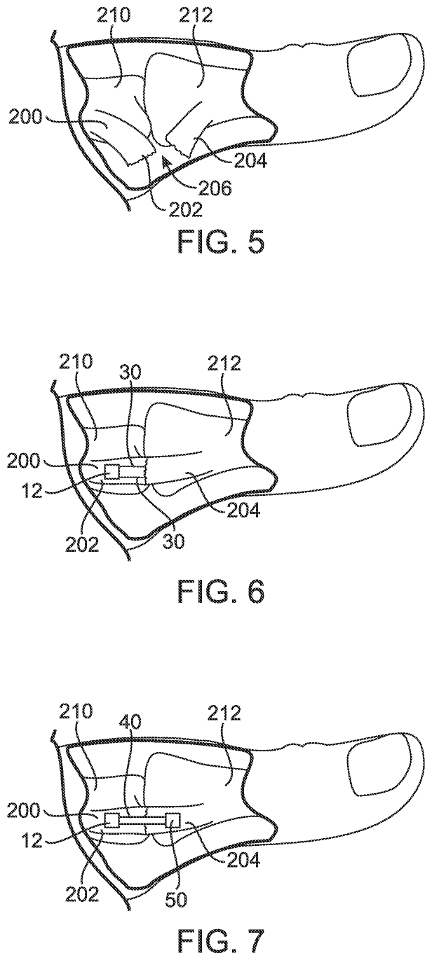

FIGS. 5-7 illustrate a method for repairing a damaged ligament 200, for example a hand ligament, having a proximal end 202 separated from a distal end 204. Referring now to FIG. 5, the proximal end 202 of the ligament 200 is secured to a proximal bone 210, and the distal end 204 of the ligament 200 is secured to a distal bone 212. A tear 206 is formed between the proximal end 202 and the distal end 204. In the exemplary embodiment, the method will be described with respect to securing the primary anchor 12 to the proximal bone 210 and proximal end 202 of the ligament 200, and securing the secondary anchor 50 to the distal bone 212 and the distal end 204 of the ligament 200; however, it will be appreciated that the primary anchor 12 may be secured to the distal bone 212 and the distal end 204 of the ligament 200, and the secondary anchor 50 may be secured to the proximal bone 210 and proximal end 202 of the ligament 200.

Referring to FIG. 6, the primary anchor 12 is positioned on the proximal bone 210 over the proximal end 202 of the damaged ligament 200. Using the pliers 100, a first arm 102 of the pliers 100 is positioned on the proximal bone 210, and an second arm 104 of the pliers 100 is positioned on the top surface 24 of the primary anchor 12. The arms 102, 104 of the pliers 100 are squeezed together to apply pressure to the primary anchor 12 so that the primary anchor tine 20 embeds within the proximal bone 210. The primary anchor 12 is secured in the proximal bone 210 so that the front end 16 faces the distal bone 212. Accordingly, the sutures 30 and the suture tape 40 extend toward the distal bone 212.

In one embodiments, the proximal end 202 and the distal end 204 of the ligament 200 are pulled together and sutured with the sutures 30. That is, the needles 34 are utilized to thread the sutures 30 through the proximal end 202 and the distal end 204 of the ligament 200 to secure the proximal end 202 and the distal end 204 of the ligament 200 together. It will be appreciated that in some embodiments, the surgical kit 10 may not include the sutures 30 and the sutures 30 may not be used to secure the proximal end 202 and the distal end 204 of the ligament 200 together. For example, the ligament 200 may only have a partial tear and may not require suturing.

Referring now to FIG. 7, the secondary anchor 50 is positioned on the distal bone 212 over the distal end 204 of the damaged ligament 200. The secondary anchor 50 is positioned so that the front end 54 of the secondary anchor 50 faces the proximal bone 210. That is, the front end 54 of the secondary anchor 50 faces the primary anchor 12. Accordingly, the opening 80 of the secondary anchor 50 faces the primary anchor 12. The free end 42 of the suture tape 40 is inserted into the opening 80 through the slot 92. The suture tape 40 is pulled through the opening 80 until the suture tape 40 is secured against the damaged ligament 200.

Using the pliers 100, the first arm 102 of the pliers 100 is positioned on the distal bone 212, and the second arm 104 of the pliers 100 is positioned on the top surface 64 of the secondary anchor 50. The arms 102, 104 of the pliers 100 are squeezed together to apply pressure to the secondary anchor 50 so that the secondary anchor tine 60 embeds within the distal bone 212. Concurrently, the inner tine 90 embeds in the free end 42 of the suture tape 40 that has been inserted in the opening 80 to secure the suture tape 40 in the secondary anchor 50. In some embodiments, the top wall 82 collapses toward the bottom wall 84 to collapse the opening 80 so that the inner tine 90 embeds into the suture tape 40.

With the suture tape 40 secure across the damaged ligament 200, the surgical site is closed with the primary anchor 12 and the secondary anchor 50 positioned in the surgical site. In some embodiments, the primary anchor 12 and the secondary anchor 50 are formed from a biocompatible material that allows the primary anchor 12 and the secondary anchor 50 to be permanently retained in the surgical site. In other embodiments, the primary anchor 12 and the secondary anchor 50 are formed from a material that absorbs into the body after surgery. For example, the primary anchor 12 and the secondary anchor 50 may be biodegradable and/or bio-absorbable. In some embodiments, the primary anchor 12 and the secondary anchor 50 are formed from a biocompatible material that allows tissue ingrowth. The primary anchor 12 and the secondary anchor 50 can be formed in different sizes and shapes to accommodate patient anatomy and pathology.

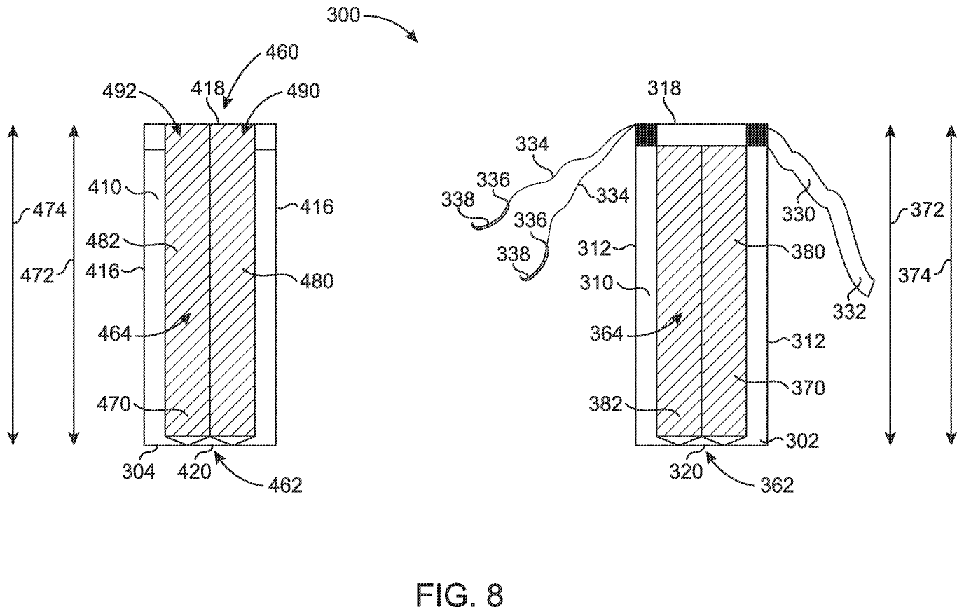

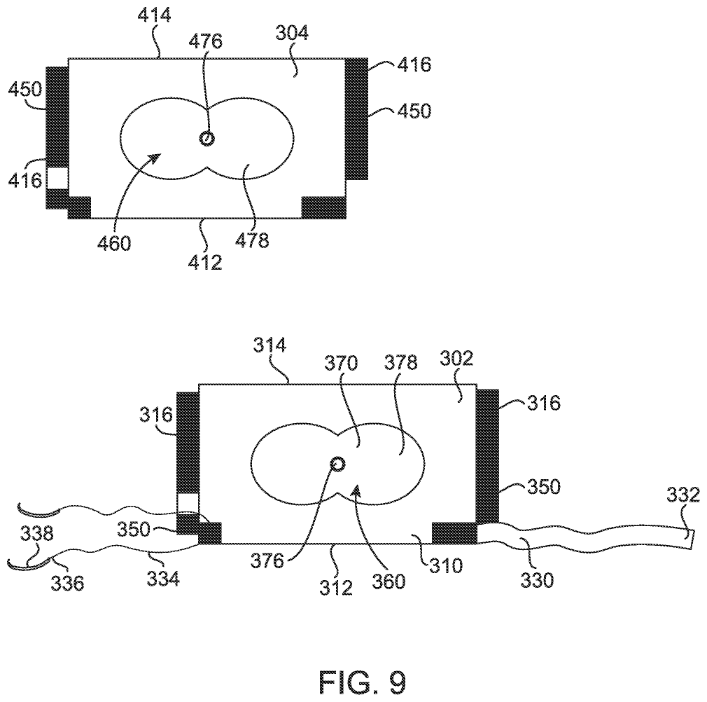

Referring now to FIGS. 8 and 9 a surgical kit 300 for repairing a damaged ligament includes a primary anchor 302 and a secondary anchor 304. The primary anchor 302 is configured to be secured to a first bone during a surgical procedure to repair the damaged ligament. The primary anchor 302 includes a body 310 having a front end 312 and an opposite back end 314. A pair of sides 316 extend between the front end 312 and the back end 314. The body 310 extends between a top 318 and a bottom 320.

Suture tape 330 is pre-attached to the primary anchor 302 prior to the surgical procedure. The suture tape 330 is attached to the front end 312 of the primary anchor 302 and extends from the primary anchor 302 to a free end 332. During surgery to repair the damaged ligament, the suture tape 330 is configured to extend over the damaged ligament. At least one suture 334 also extends from the primary anchor 302. The at least one suture extends from the front end 312 of the primary anchor 302 to a free end 336. A needle 338 is attached to the free end 336. The at least one suture 334 is configured to suture a first end of the damaged ligament to a second end of the damaged ligament during the surgical procedure.

A pair of flanges 350 extend from each side 316 of the primary anchor 302. The flanges 350 are generally rounded and configured to deform as the primary anchor 302 is secured to the first bone.

The body 310 of the primary anchor 302 includes a top opening 360 formed in the top 318 of the body 310. An opposite bottom opening 362 is formed in the bottom 320 of the body 310. A channel 364 extends through the body 310 from the top opening 360 to the bottom opening 362. A first inner bone anchor 370 extends through the channel 364. The first inner bone anchor 370 has a length 372 that is substantially the same as a length 374 of the body 310. Accordingly, the first inner bone anchor 370 extends from the top opening 360 to a bottom opening 362 so that the first inner bone anchor 370 extends from the top 318 of the body 310 to the bottom 320 of the body 310. The first inner bone anchor 370 is configured to move from a disengaged position within the body 310 to an engaged position, wherein the first inner bone anchor 370 is pushed through the bottom opening 362 and bends outward. A threaded opening 376 is positioned at a top 378 of the first inner bone anchor 370.

In the illustrated embodiment, the first inner bone anchor 370 includes a pair of first inner bone anchors 370. The pair of first inner bone anchors 370 includes a right side inner anchor 380 and a left side inner anchor 382. The right side inner anchor 380 and a left side inner anchor 382 are positioned adjacent to one another in the channel 364. The right side inner anchor 380 and a left side inner anchor 382 are configured to bend outward in opposite directions into the first bone when the first inner bone anchor 370 is moved to the engaged position.

The first inner bone anchor 370 can be made of metal, polymer, plastic, etc. In some embodiments, the first inner bone anchor 370 is a memory anchor that hooks outward via the memory when the primary anchor 302 is secured to the bone. The first inner bone anchor 370 can have dynamic anchoring capabilities that are designed to facilitate bone attachment of ligament and other soft tissues. The first inner bone anchor 370 can come in varying widths, lengths, and sizes, allowing fixation in most procedures. The first inner bone anchor 370 can have sharp ends that assist with anchoring to the inner bone. The first inner bone anchor 370 extends out fully unfolded laterally and medially to anchor to the inner bone.

The secondary anchor 304 is configured to be secured to a second bone during a surgical procedure to repair the damaged ligament. The secondary anchor 304 includes a body 410 having a front end 412 and an opposite back end 414. A pair of sides 416 extend between the front end 412 and the back end 414. The body 410 extends between a top 418 and a bottom 420.

A pair of flanges 450 extend from each side 416 of the secondary anchor 304. The flanges 450 are generally rounded and configured to deform as the secondary anchor 304 is secured to the first bone.

The body 410 of the secondary anchor 304 includes a top opening 460 formed in the top 418 of the body 410. An opposite bottom opening 462 is formed in the bottom 420 of the body 410. A channel 464 extends through the body 410 from the top opening 460 to the bottom opening 462. A second inner bone anchor 470 extends through the channel 464. The second inner bone anchor 470 has a length 472 that is substantially the same as a length 474 of the body 410. Accordingly, the second inner bone anchor 470 extends from the top opening 460 to a bottom opening 462 so that the second inner bone anchor 470 extends from the top 418 of the body 410 to the bottom 420 of the body 410. The second inner bone anchor 470 is configured to move from a disengaged position within the body 410 to an engaged position, wherein the second inner bone anchor 470 is pushed through the bottom opening 462 and bends outward. A threaded opening 476 is positioned at a top 478 of the second inner bone anchor 470.

In the illustrated embodiment, the second inner bone anchor 470 includes a pair of second inner bone anchors 470. The pair of second inner bone anchors 470 includes a right side inner anchor 480 and a left side inner anchor 482. The right side inner anchor 480 and a left side inner anchor 482 are positioned adjacent to one another in the channel 464. The right side inner anchor 480 and a left side inner anchor 482 are configured to bend outward in opposite directions into the second bone when the second inner bone anchor 470 is moved to the engaged position.

The second inner bone anchor 470 can be made of metal, polymer, plastic, etc. In some embodiments, the second inner bone anchor 470 is a memory anchor that hooks outward via the memory when the primary anchor 302 is secured to the bone. The second inner bone anchor 470 can have dynamic anchoring capabilities that are designed to facilitate bone attachment of ligament and other soft tissues. The second inner bone anchor 470 can come in varying widths, lengths, and sizes, allowing fixation in most procedures. The second inner bone anchor 470 can have sharp ends that assist with anchoring to the inner bone. The second inner bone anchor 470 extends out fully unfolded laterally and medially to anchor to the inner bone.

An opening 490 is formed in the front end 412 of the body 410 adjacent the top 418 of the body 410. A cavity 492 extends from the opening 490 into the body 492. The cavity 492 may extend from the front end 412 to the back end 414 of the body 410. During surgery, the front end 312 of the primary anchor 302 is positioned to face the front end 412 of the secondary anchor 304. The cavity 492 is configured to receive the free end 332 of the suture tape 330. In some embodiments, the cavity 490 collapses to secure the suture tape 330 therein.

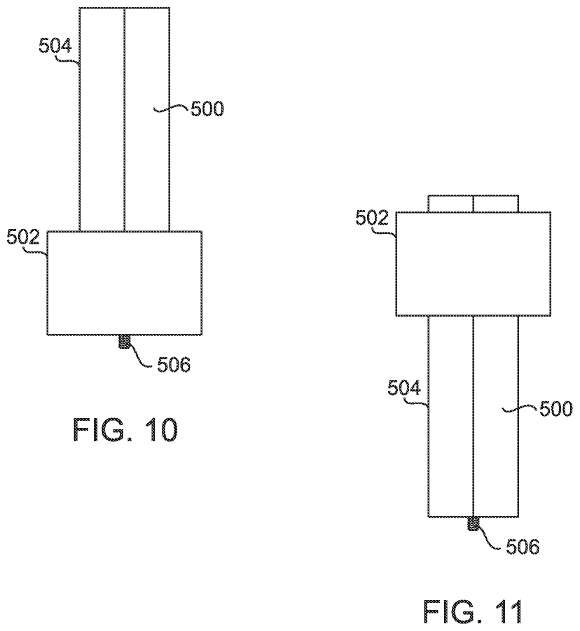

Referring to FIGS. 10 and 11, a press 500 includes a main body 502 and an actuating body 504 that extends through the main body 502. The actuating body 504 includes a thread screw 506 extending from a bottom 508 of the actuating body 504. FIG. 10 illustrates the press 500 in a disengaged position. As illustrated in FIG. 11, the actuating body 504 slides through the main body 502 to an engaged position.

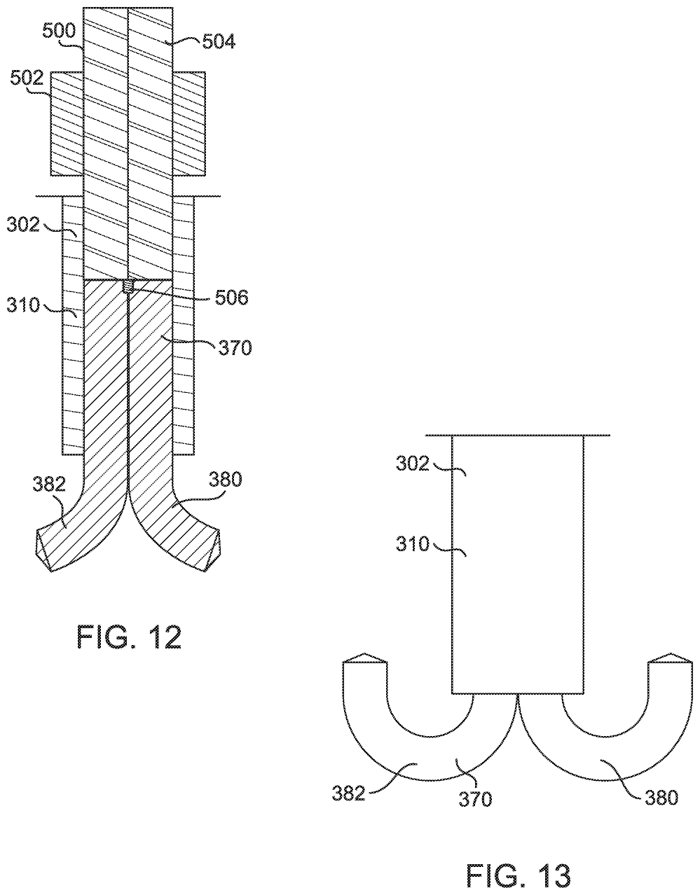

FIG. 12 is described with respect to the primary anchor 302; however, it will be appreciated that the press 500 operates in a similar manner with the secondary anchor 304. As illustrated in FIG. 12, the main body 502 of the press 500 is positioned on the top 318 of the primary anchor 302. The threaded screw 506 is secured into the threaded opening 376 of the first inner bone anchor 370. As the actuating body 504 of the press 500 is slid downward, the first inner bone anchor 370 extends from the bottom 320 of the primary anchor 302 into the engaged position to secure within the first bone. The first inner bone anchor 370 is illustrated in the engaged position in FIG. 13. In the engaged position, the flanges 350 flatten flush across the first bone and the right side inner anchor 380 and a left side inner anchor 382 flare outward and embed within the first bone to secure the primary anchor 302 to the first bone.

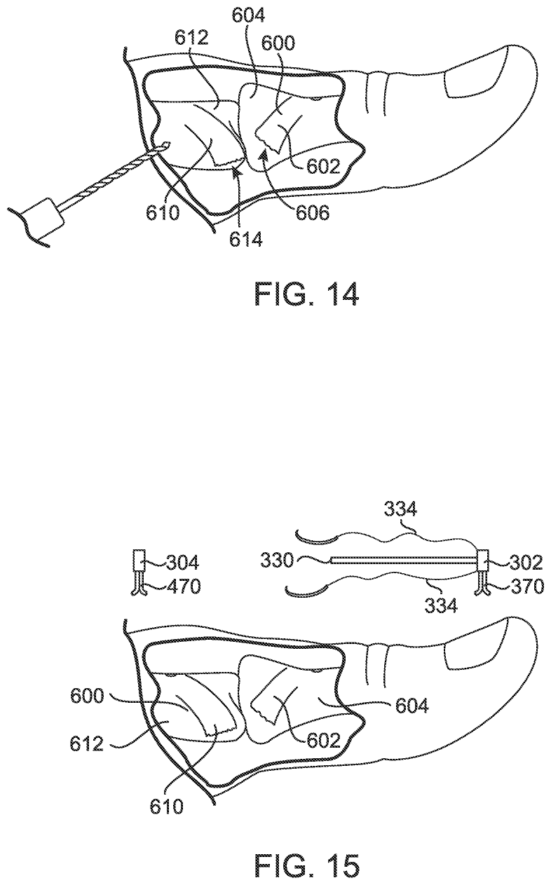

FIGS. 14-17 illustrate a method for repairing a damaged ligament 600 that includes a first segment 602 coupled to a first bone 604 and extending to a free end 606. The ligament 600 also includes a second segment 610 coupled to a second bone 612 and extending to a free end 614. The method includes drilling an opening 620 in the first bone 604 and drilling an opening 622 in the second bone 612, as illustrated in FIGS. 14 and 15.

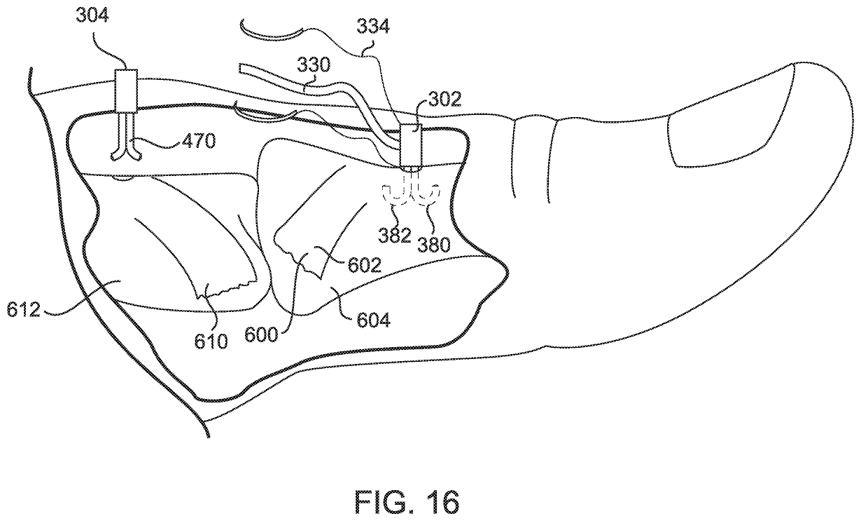

The primary anchor 302 is secured to the first bone 604 by expanding the first inner bone anchor 370 into the opening 620 using the press 500. In particular, as illustrated in FIG. 16, the first inner bone anchor 370 is engaged with the press 500 through the top opening 360 of the primary anchor 302. The threaded screw 506 is secured into the threaded opening 376 of the first inner bone anchor 370. The first inner bone anchor 370 is pushed with the press 500 at least partially through the bottom opening 362 in the primary anchor 302 so that the first inner bone anchor 370 bends outward into the first bone 604. That is, the right side inner anchor 380 and a left side inner anchor 382 flare outward and embed within the first bone 604 to secure the primary anchor 302 to the first bone 604.

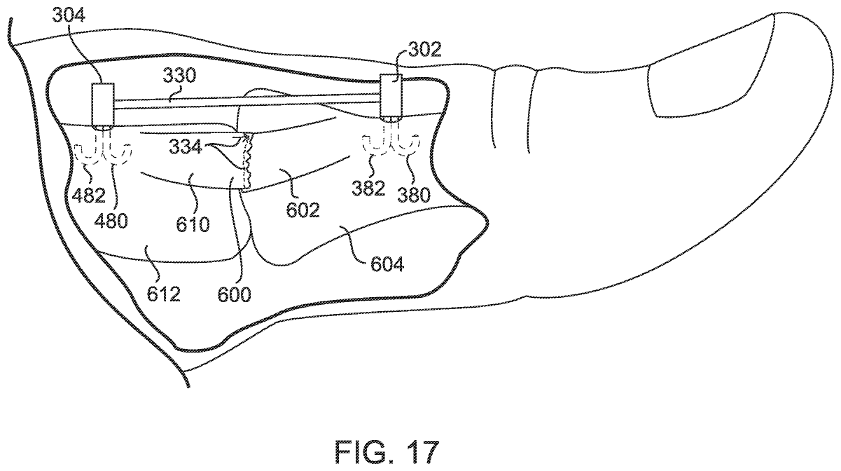

The secondary anchor 304 is secured to the second bone 612 by expanding the second inner bone anchor 470 into the opening 622 using the press 500. In particular, as illustrated in FIG. 17, the second inner bone anchor 470 is engaged with the press 500 through the top opening 460 of the secondary anchor 304. The threaded screw 506 is secured into the threaded opening 476 of the second inner bone anchor 470. The second inner bone anchor 470 is pushed with the press 500 at least partially through the bottom opening 462 in the secondary anchor 304 so that the second inner bone anchor 470 bends outward into the second bone 612. That is, the right side inner anchor 480 and a left side inner anchor 482 flare outward and embed within the second bone 612 to secure the secondary anchor 304 to the second bone 612.

The first segment 602 of the damaged ligament 600 is sutured to the second segment 610 of the damaged ligament 600 with the at least one suture 334 extending from the primary anchor 302. Next, the suture tape 330 is extended over the damaged ligament 600 and the free end 332 of the suture tape 330 is secured in the cavity 492 of the secondary anchor 304. With the suture tape 330 secure across the damaged ligament 600, the surgical site is closed with the primary anchor 302 and the secondary anchor 304 positioned in the surgical site. In some embodiments, the primary anchor 302 and the secondary anchor 304 are formed from a biocompatible material that allows the primary anchor 302 and the secondary anchor 304 to be permanently retained in the surgical site. In other embodiments, the primary anchor 302 and the secondary anchor 304 are formed from a material that absorbs into the body after surgery. For example, the primary anchor 302 and the secondary anchor 304 may be biodegradable and/or bio-absorbable. In some embodiments, the primary anchor 302 and the secondary anchor 304 are formed from a biocompatible material that allows tissue ingrowth. The primary anchor 302 and the secondary anchor 304 can be formed in different sizes and shapes to accommodate patient anatomy and pathology.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles can have been presented, they need not be utilized in combination, and many combinations of aspects and principles are possible in light of the various embodiments provided above.

1. A surgical kit for repairing a damaged ligament, the surgical kit comprising:
a first anchor configured to be secured to a first bone during a surgical procedure to repair the damaged ligament, wherein the first anchor includes a first inner bone anchor that is secured in an opening drilled in the first bone,
suture tape pre-attached to the first anchor prior to the surgical procedure and extending from the first anchor to a free end, the suture tape configured to extend over the damaged ligament, and
a second anchor configured to secure the free end of the suture tape to a second bone adjacent the first bone during the surgical procedure.

2. The surgical kit of claim 1, wherein the first inner bone anchor extends from a top opening in the first anchor to a bottom opening in the first anchor.

3. The surgical kit of claim 2, further comprising a press configured to engage the first inner bone anchor through the top opening in the first anchor, wherein the press is configured to push the first inner bone anchor at least partially through the bottom opening in the first anchor.

4. The surgical kit of claim 3, wherein the first inner bone anchor bends outward into the first bone as the first inner bone anchor is pushed through the bottom opening in the first anchor.

5. The surgical kit of claim 4, wherein the first inner bone anchor includes two first inner bone anchors, wherein the two first inner bone anchors bend outward in opposite directions into the first bone as the two first inner bone anchors are pushed through the bottom opening in the first anchor.

6. The surgical kit of claim 3, wherein the first inner bone anchor includes a threaded opening and the press includes a threaded screw, wherein the threaded screw of the press is secured within the threaded opening of the first inner bone anchor.

7. The surgical kit of claim 1, wherein the second anchor includes a second inner bone anchor that is secured in an opening drilled in the second bone.

8. The surgical kit of claim 1, further comprising at least one suture extending from the first anchor, wherein the at least one suture is configured to suture a first end of the damaged ligament to a second end of the damaged ligament during the surgical procedure.

9. The surgical kit of claim 1, wherein the second anchor further comprises an opening configured to receive the free end of the suture tape.

10. A method for repairing a damaged ligament, the method comprising:
drilling an opening into a first bone during a surgical procedure to repair the damaged ligament,
securing a first anchor to a first bone by securing a first inner bone anchor of the first anchor in the opening drilled in the first bone,
extending suture tape over the damaged ligament, wherein the suture tape is pre-attached to the first anchor prior to the surgical procedure and extends from the first anchor to a free end, and
securing the free end of the suture tape to a second bone adjacent the first bone during the surgical procedure with a second anchor.

11. The method of claim 10, further comprising:
engaging the first inner bone anchor through a top opening in the first anchor with a press, and
pushing the first inner bone anchor at least partially through a bottom opening in the first anchor with the press.

12. The method of claim 11, further comprising pushing the first inner bone anchor through the bottom opening in the first anchor so that the first inner bone anchor bends outward into the first bone.

13. The method of claim 12, wherein the first inner bone anchor includes two first inner bone anchors, and the method further comprises pushing the two first inner bone anchors through the bottom opening in the first anchor so that the two first inner bone anchors bend outward in opposite directions into the first bone.

14. The method of claim 1, further comprising securing a threaded screw of the press within a threaded opening of the first inner bone anchor.

15. The method of claim 10, further comprising securing the second anchor to the second bone by securing a second inner bone anchor of second anchor in an opening drilled in the second bone.

16. The method of claim 10, further comprising suturing a first end of the damaged ligament to a second end of the damaged ligament with at least one suture extending from the first anchor during the surgical procedure.

17. The method of claim 10, further comprising positioning the free end of the suture tape in an opening of the second anchor.

18. A surgical anchor for repairing a damaged ligament, the anchor comprising:
an anchor body having a top and a bottom, wherein the anchor body is positioned on a bone during a surgical procedure to repair the damaged ligament with the bottom of the anchor body positioned against the bone,
a top opening formed in the top of the anchor body,
a bottom opening formed in a bottom of the anchor body,
a channel extending from the top opening to the bottom opening, and
an inner bone anchor extending through the channel,
wherein the inner bone anchor is configured to be pushed at least partially through the bottom opening into the bone by a press that engages the inner bone anchor through the top opening.

19. The surgical anchor of claim 18, wherein the inner bone anchor bends outward into the bone as the inner bone anchor is pushed through the bottom opening in the anchor body.

20. The surgical anchor of claim 18, further comprising suture tape pre-attached to the anchor body prior to the surgical procedure and extending from the anchor body to a free end, the suture tape configured to extend over the damaged ligament.
